(19) United States
(12) Patent Application Publication
(10) Pub. No.: US 2023/0255912 A1
(43) Pub. Date: Aug. 17, 2023

(54) COMPOSITIONS AND METHODS FOR TREATING CRP-MEDIATED DISEASES

(71) Applicant: Ponce de Leon Health Designated Activity Company, Dorgheda (IE)

(72) Inventor: David Eugene PEREIRA, Salem, VA (US)

(21) Appl. No.: 18/013,414

(22) PCT Filed: Jul. 2, 2021
(86) PCT No.: PCT/US2021/040257

§ 371 Date: Dec. 28, 2022

Related U.S. Application Data
(60) Provisional application No. 63/047,302, filed on Jul. 2, 2020.

(51) Int. Cl. A61K 31/194; A61K 31/07; A61K 31/593; A61K 9/00; A61P 37/02

(52) U.S. Cl. CPC A61K 31/194; A61K 31/07; A61K 31/593; A61K 9/0053; A61P 37/02

(57) ABSTRACT

Described herein are compositions, and methods for treating diseases, illness, and symptoms associated with C-reactive protein (CRP) production and dysfunction.

### TECHNICAL FIELD

The invention described herein pertains to compounds, compositions, and methods for treating diseases, illness, and symptoms associated with C-reactive protein (CRP) production and dysfunction.

### BACKGROUND AND SUMMARY OF THE INVENTION

In 2015 the US Census Bureau estimated that 617.1 million people worldwide were over the age of 65 years or 8.5% of the world's population. It is expected that the percentage of the world's population over 65 years old will continue to grow reaching 1.6 billion older adults by 2050 (16.7% of the world's population). At the same time, it has been reported that the number of people below the age of 20 will remain constant. This transition to an older population is expected to result in an increased burden to society due to age-related diseases and disabilities. One approach to ease the burden of an aging population is to expand the healthspan of the elderly. Healthspan can be defined as the period of life spent in good health, free from the chronic diseases and disabilities related to aging. Healthspan may be positively impacted by the treatment or moderation of one or more aging processes, such as metabolism, macromolecular damage, epigenetics, inflammation, adaption to stress, proteostasis, and the like.

It has been unexpectedly discovered that the compounds described herein are capable of decreasing circulating levels of C-reactive protein (CRP). Without being bound by theory, it is believed that the compounds and compositions described herein elicit their therapeutic effects on age-related diseases by modulating C-reactive protein (CRP) expression and/or signaling, and/or IL-6 expression and/or signaling, or both.

It has also been discovered that the compounds, compositions, and methods described herein are useful for treating a variety of age-related diseases. Illustrative age-related disease include, but are not limited to, musculoskeletal decline, systemic inflammation, also known as chronic inflammation, and cognitive decline. Illustrative examples of musculoskeletal decline include, but are not limited to, muscle atrophy, sarcopenia, and the like.

In one illustrative embodiment of the invention, compositions comprising AKG and/or salts thereof are described herein for treating age-related diseases. The compositions include a therapeutically effective amount of AKG and/or one or more salts thereof. It is to be understood that the compositions may include other components and/or ingredients, including, but not limited to, other therapeutically active compounds, and/or one or more carriers, diluents, excipients, and the like, and combinations thereof.

In another embodiment, methods for treating host animals with age-related diseases are also described herein, where the methods include administering a therapeutically effective amount of one or more of the compounds and/or compositions described herein to the host animal. In another embodiment, uses of the compounds and compositions in the manufacture of a medicament useful or adapted for treating host animals with age-related diseases are also described herein. In another embodiment, the medicaments include a therapeutically effective amount of the one or more compounds and/or compositions for treating a host animal with age-related diseases.

It is to be understood herein that the compounds and compositions, and methods and uses described herein may be used alone or in combination with other compounds and/or compositions useful for treating age-related diseases, including those compounds that may be therapeutically effective by the same or different modes of action. In addition, it is to be understood herein that the compounds described herein may be used in combination with other compounds that are administered to treat other symptoms of age-related diseases.

### DETAILED DESCRIPTION

Several illustrative embodiments of the invention are described by the following enumerated clauses:

1. A composition for treating a CRP mediated disease in a host animal, the composition comprising a therapeutically effective amount of AKG, one or more salts thereof, or a combination of any of the foregoing, and optionally one or more diluents, excipients, or carriers, or a combination of any of the foregoing.

2. The composition of clause 1 wherein the CRP mediated disease is musculoskeletal decline.

3. The composition of clause 1 wherein the CRP mediated disease is a muscle atrophy, also known as muscle wasting and muscle loss disease.

4. The composition of clause 1 wherein the CRP mediated disease is sarcopenia.

5. The composition of clause 1 wherein the CRP mediated disease is a systemic inflammation.

6. The composition of clause 1 wherein the CRP mediated disease is a cardiovascular disease associated with systemic inflammation.

7. The composition of clause 1 wherein the CRP mediated disease is acute coronary syndrome associated with systemic inflammation.

8. The composition of clause 1 wherein the CRP mediated disease is hemorrhagic stroke associated with systemic inflammation.

9. The composition of clause 1 wherein the CRP mediated disease is age-related macular degeneration (AMD) associated with systemic inflammation.

10. The composition of clause 1 wherein the CRP mediated disease is type 2 diabetes mellitus associated with systemic inflammation.

11. The composition of clause 1 wherein the CRP mediated disease is fatty liver disease associated with systemic inflammation.

12. The composition of clause 1 wherein the CRP mediated disease is fibrosis, including fibrosis associated with fatty liver disease.

13. The composition of clause 1 wherein the CRP mediated disease includes excessive cytokine signaling.

14. The composition of clause 13 wherein the cytokines include one or more of IL3, IL6, IL-7, TNFα, or MIP-1β, or a combination thereof

15. The composition of clause 1 wherein the CRP mediated disease is inflammaging.

16. The composition of clause 1 wherein the CRP mediated disease is cognitive decline.

17. The composition of clause 1 wherein the CRP mediated disease is cognitive decline associated with a neurodegenerative disease, such as Alzheimer's disease, Parkinson's disease, and Huntington's disease.

The composition of any one of the preceding clauses wherein the therapeutically effective amount is an amount capable of decreasing circulating levels of CRP in the host animal.

The composition of any one of the preceding clauses wherein the therapeutically effective amount is a daily dose in the range from about 100 mg to about 3,500 mg of AKG, which is optionally divided.

The composition of any one of the preceding clauses wherein the therapeutically effective amount is a daily dose in the range from about 200 mg to about 3,500 mg of AKG, which is optionally divided.

The composition of any one of the preceding clauses wherein the therapeutically effective amount is a daily dose in the range from about 250 mg to about 3,500 mg of AKG, which is optionally divided.

The composition of any one of the preceding clauses wherein the therapeutically effective amount is a daily dose in the range from about 250 mg to about 3,000 mg of AKG, which is optionally divided.

The composition of any one of the preceding clauses wherein the therapeutically effective amount is a daily dose in the range from about 250 mg to about 2,500 mg of AKG, which is optionally divided.

The composition of any one of the preceding clauses wherein the therapeutically effective amount is a daily dose in the range from about 250 mg to about 2,000 mg of AKG, which is optionally divided.

The composition of any one of the preceding clauses wherein the therapeutically effective amount is a daily dose in the range from about 250 mg to about 1,500 mg of AKG, which is optionally divided.

The composition of any one of the preceding clauses wherein the therapeutically effective amount is a daily dose in the range from about 250 mg to about 1,250 mg of AKG, which is optionally divided.

The composition of any one of the preceding clauses wherein the therapeutically effective amount is a daily dose in the range from about 250 mg to about 1,000 mg of AKG, which is optionally divided.

The composition of any one of the preceding clauses wherein the therapeutically effective amount is a daily dose in the range from about 250 mg to about 750 mg of AKG, which is optionally divided.

The composition of any one of the preceding clauses wherein the therapeutically effective amount is a daily dose in the range from about 300 mg to about 3,500 mg of AKG, which is optionally divided.

The composition of any one of the preceding clauses wherein the therapeutically effective amount is a daily dose in the range from about 300 mg to about 3,000 mg of AKG, which is optionally divided.

The composition of any one of the preceding clauses wherein the therapeutically effective amount is a daily dose in the range from about 300 mg to about 2,500 mg of AKG, which is optionally divided.

The composition of any one of the preceding clauses wherein the therapeutically effective amount is a daily dose in the range from about 300 mg to about 2,000 mg of AKG, which is optionally divided.

The composition of any one of the preceding clauses wherein the therapeutically effective amount is a daily dose in the range from about 300 mg to about 1,500 mg of AKG, which is optionally divided.

The composition of any one of the preceding clauses wherein the therapeutically effective amount is a daily dose in the range from about 300 mg to about 1,250 mg of AKG, which is optionally divided.

The composition of any one of the preceding clauses wherein the therapeutically effective amount is a daily dose in the range from about 300 mg to about 1,000 mg of AKG, which is optionally divided.

The composition of any one of the preceding clauses wherein the therapeutically effective amount is a daily dose in the range from about 300 mg to about 750 mg of AKG, which is optionally divided.

The composition of any one of the preceding clauses wherein the therapeutically effective amount is a daily dose in the range from about 350 mg to about 3,500 mg of AKG, which is optionally divided.

The composition of any one of the preceding clauses wherein the therapeutically effective amount is a daily dose in the range from about 350 mg to about 3,000 mg of AKG, which is optionally divided.

The composition of any one of the preceding clauses wherein the therapeutically effective amount is a daily dose in the range from about 350 mg to about 2,500 mg of AKG, which is optionally divided.

The composition of any one of the preceding clauses wherein the therapeutically effective amount is a daily dose in the range from about 350 mg to about 2,000 mg of AKG, which is optionally divided.

The composition of any one of the preceding clauses wherein the therapeutically effective amount is a daily dose in the range from about 350 mg to about 1,500 mg of AKG, which is optionally divided.

The composition of any one of the preceding clauses wherein the therapeutically effective amount is a daily dose in the range from about 350 mg to about 1,250 mg of AKG, which is optionally divided.

The composition of any one of the preceding clauses wherein the therapeutically effective amount is a daily dose in the range from about 350 mg to about 1,000 mg of AKG, which is optionally divided.

The composition of any one of the preceding clauses wherein the therapeutically effective amount is a daily dose in the range from about 350 mg to about 750 mg of AKG, which is optionally divided.

The composition of any one of the preceding clauses The composition of any one of the preceding clauses wherein the composition comprises CaAKG.

It is to be understood herein that amounts of AKG refer to the net amount of the diacid, and do not include hydrations states nor counterions such as sodium, potassium, calcium, zinc, and the like. For example, compositions described herein comprising about 750 mg of AKG where the AKG is included in the composition as the CaAKG salt, such compositions comprise about 925 mg to about 975 mg of CaAKG. Similarly, compositions described herein comprising about 750 mg of AKG where the AKG is included in the composition as the monohydrate, such compositions comprise about 825 mg to about 875 mg of the monohydrate. Similarly, compositions described herein comprising about 750 mg of AKG where the AKG is included in the composition as the CaAKG monohydrate, such compositions comprise about 1,025 mg to about 1,075 mg of CaAKG monohydrate.

The composition of any one of the preceding clauses further comprising one or more vitamins.

The composition of any one of the preceding clauses further comprising vitamin A.

The composition of the preceding clause wherein the 1,000-fold weight ratio of vitamin A/AKG is in the range from about 0.5 to about 1.5; from about 0.6 to about 1.4, from about 0.7 to about 1.3, from about 0.8 to about 1.2, or from about 0.85 to about 1. where the 1,000-fold weight ratio of vitamin A/AKG is about 0.9.

The composition of any one of the preceding clauses further comprising vitamin D.

The composition of the preceding clause wherein the 1,000-fold weight ratio of vitamin D/AKG is in the range from about 0.005 to about 0.06, from about 0.01 to about 0.06, from about 0.01 to about 0.05, from about 0.015 to about 0.05, from about 0.015 to about 0.045, from about 0.015 to about 0.04, from about 0.02 to about 0.04, from about 0.02 to about 0.035, or from about 0.02 to about 0.03. where the 1,000-fold weight ratio of vitamin D/AKG is about 0.025.

A method for treating a CRP mediated disease in a host animal, the method comprising administering the composition of any one of the preceding clauses to the host animal.

Use of the composition of any one of the preceding clauses in the manufacture of a medicament for treating a CRP mediated disease in a host animal.

A method for treating a CRP mediated disease in a host animal, the method comprising administering a therapeutically effective amount of AKG, one or more salts thereof, or a combination of any of the foregoing, and optionally one or more diluents, excipients, or carriers, or a combination of any of the foregoing to the host animal.

Use of a therapeutically effective amount of AKG, one or more salts thereof, or a combination of any of the foregoing, and optionally one or more diluents, excipients, or carriers, or a combination of any of the foregoing in the manufacture of a medicament for treating a CRP mediated disease in a host animal.

Alpha-ketoglutarate (AKG) or α-ketoglutarate (Formula 1) is also known as 2-oxopentanedioic acid, 2-ketoglutaric acid, 2-oxoglutaric acid, and oxoglutaric acid. At physiological pH, AKG exists in one or more deprotonated forms, such as those depicted as Formulae 2.

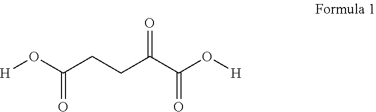

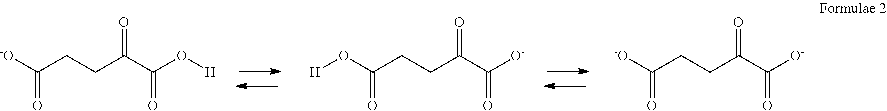

AKG and AKG salts are grandfathered as GRAS compounds because these compounds were supplements prior to the Dietary Supplement Health and Education Act of 1994 (“DSHEA”). The toxicological profile of AKG has been evaluated and shown to be non-toxic. No serious adverse events were reported in clinical trials related to AKG or CaAKG. Both AKG and its corresponding salts are commercially available, either via preparation from fermentation cultures (for example see U.S. Pat. No. 2,776,926) or chemical synthesis from closely related compounds. AKG and salts thereof are currently sold in the United States as supplements.

AKG is an endogenous metabolite found in all aerobic organisms including humans. It was identified in the 1930's as a metabolite in the Krebs cycle (also known as the citric acid cycle (CAC) and tricarboxylic acid (TCA) cycle). The Krebs cycle is a series of enzymatic reactions that produce energy for the cell; these enzymes are found in the cellular mitochondria. AKG is an intermediate in the Krebs cycle of eukaryotic organisms and is biosynthesized from isocitrate (in the Krebs cycle process) or L-glutamate (via alanine transaminase) in such organisms. Consistent with its role in energy generation via the Krebs cycle, AKG is an important regulator of bioenergetics in cells and is implicated as an inhibitor of ATP synthase subunit 13 and an indirect inhibitor of the kinase mTOR, a consequence of partial inhibition of the mitochondrial electron transport chain. Besides their role in ATP production (cellular energy), Krebs cycle metabolites, including AKG, play a role in amino acid synthesis, NAD/NADH generation and other cellular biochemical processes. For example, three enzymes in the Krebs cycle—citrate synthase (CS), a-ketoglutarate dehydrogenase (KGDH), and malate dehydrogenase (MDH)—decrease in activity during ageing. Without being bound by theory, it is believed herein that the decreased metabolic function in each case contributes to organism aging. In addition, the plasma level of AKG may decrease by a factor of 10 in humans between the age of 40 and 80.

It has been discovered herein that AKG and salts thereof decrease inflammation. Without being bound by theory it is believed herein that AKG and salts thereof treat inflammation by modulating C-reactive protein (CRP) expression and/or signaling, and/or IL-6 expression and/or signaling, or both.

It has also been discovered herein that AKG and salts thereof decrease musculoskeletal decline. Without being bound by theory it is believed herein that AKG and salts thereof treat musculoskeletal decline by modulating C-reactive protein (CRP) expression and/or signaling.

It has also been discovered herein that AKG and salts thereof decrease cognitive decline. Without being bound by theory it is believed herein that AKG and salts thereof treat cognitive decline by modulating C-reactive protein (CRP) expression and/or signaling.

AKG is generally not available in the typical human diet, and therefore, an AKG based dietary supplement can provide AKG to the human diet. Therefore, dietary supplementation of AKG (for example, in the form of calcium alpha-ketoglutarate monohydrate) may be able to compensate for the decrease of AKG levels during aging and result in improvement of aging processes such as a decreasing systemic inflammation, musculoskeletal decline, and/or cognitive decline as measured by CRP.

Inflammatory biomarkers, such as C-reactive protein (CRP) or IL-6, have been used to assess the level of inflammation during aging. The levels of IL-6 and CRP reportedly increase in an age-depended manner. IL-6 and CRP levels were also found to correlate with physical and cognitive abilities in participants with aging-related health issues and in participants aging normally (a group categorized as “aging successfully”). Those individuals with elevated IL-6 and/or CRP performed worse on cognitive and performance tests and had a greater risk of death. Without being bound by theory, it is believed herein that IL-6 and CRP are not only markers that can be used to assess aging status in adults, but also play a causative role in aging.

The population can generally be divided into groups characterized by stable low level CRP; stable high level CRP, medium moving to high level CRP; and high moving to low level CRP. The stable low level group is categorized as healthy aging defined as having an absence of chronic disease (coronary heart disease, stroke, diabetes, and cancer), disability, depressive symptoms, and more favorable levels of memory, respiratory function, blood pressure, and walking speed. Likewise, the high-to-medium group is associated with healthier lifestyle and improved/stabilized cardiometabolic profile. The stable high and medium to high CRP level groups are characterized by having unfavorable physical and cognitive scores resulting in less healthy aging.

In another embodiment, methods for treating and/or decreasing systemic inflammation, also known as chronic inflammation, are described herein. It has been reported that systemic inflammation can lead to a wide range of diseases, including but not limited to cardiovascular disease, hypertension, hypertensive cardiovascular and kidney complications, diabetic nephropathy, acute and chronic kidney diseases, fibrosis in cardiovascular and kidney diseases, diabetes mellitus, fatty liver diseases, liver fibrosis, and the like. It is understood herein that decreasing systemic inflammation may have numerous downstream benefits, including decreasing the likelihood of a host animal's developing, cardiovascular disease, coronary heart disease, cancer, diabetes mellitus, chronic kidney disease, fatty liver disease, including non-alcoholic steatohepatitis (NASH), fibrosis, including liver fibrosis, autoimmune disorders, and neurodegenerative disorders. In addition, in those host animals that already suffer from the disease, the compounds, compositions, and methods described herein may be used to decrease the progression of any of the foregoing diseases. It is also understood that the compounds, compositions, and methods described herein may be used as a second-line therapy in conjunction with other first-line therapies for treating any of the foregoing diseases. It is believed herein that the compounds and compositions described herein are capable of decreasing CRP levels, and thereby, preventing, slowing the progression of, or treating the numerous diseases that result from systemic inflammation.

Diabetes mellitus (DM), commonly referred to as diabetes, is a group of metabolic diseases characterized by prolonged periods of high blood sugar levels. If left untreated, diabetes can cause many complications, including acute complications such as diabetic ketoacidosis and nonketotic hyperosmolar coma, and serious long-term complications such as cardiovascular disease, stroke, kidney failure, foot ulcers and damage to the eyes. Without being bound by theory, it is believed herein that diabetes disease progression is exacerbated by levels of circulating CRP.

There are three main types of diabetes mellitus. Type 1 DM, also referred to as insulin-dependent diabetes mellitus (IDDM) or juvenile diabetes, results from the insufficient insulin production. Type 2 DM, also referred to as non insulin-dependent diabetes mellitus (NIDDM) or adult-onset diabetes, generally begins with insulin resistance, where cells fail to properly respond to insulin. Type 2 DM may lead to type 1 DM. Gestational diabetes, the third type, occurs when pregnant women without a previous history of diabetes develop high blood glucose levels.

An estimated 387 million people have diabetes worldwide, with type 2 diabetes accounting for about 90% of the cases. Diabetes is estimated to result in 2 to 5 million deaths per year. In addition, the number of people with diabetes is reportedly expected to continually rise year-after-year. The global economic cost of diabetes is estimated to be approaching $1 trillion.

The body obtains glucose from the intestinal absorption of food, the breakdown of glycogen stored in the liver, and gluconeogenesis, the generation of glucose from non-carbohydrate sources in the body. Insulin is the principal hormone that regulates the uptake of glucose from the blood into most cells of the body, especially liver, muscle, and adipose tissue. Insulin balances glucose levels in the body by inhibiting gluconeogenesis and/or the breakdown of glycogen. Insulin also stimulates glucose transport into fat and muscle cells, and stimulates the storage of glucose in the form of glycogen in the liver.

Thus, in cases of insulin dysfunction, glucose will not be properly absorbed or appropriately stored in the liver and muscles. The net effect is persistently high levels of blood glucose, poor protein synthesis, acidosis, and other metabolic dysfunction, including glycosuria, polyuria and increased fluid loss, lost blood volume, dehydration and polydipsia.

In many cases, diabetes is comorbid with fatty liver disease (FLD), including for example, non-alcoholic steatohepatitis (NASH). FLD, also referred to as fatty liver, is a reversible condition where large vacuoles of triglyceride fat accumulate in liver cells via the process of steatosis, an abnormal retention of lipids within a cell. FLD generally shows microvesicular and macrovesicular fatty changes at different stages. Though FLD itself may be reversible, the accumulation of fat may also be accompanied by a progressive hepatitis, inflammation of the liver, generally referred to as steatohepatitis, and lead to more severe nonalcoholic fatty liver disease (NAFLD), and the more severe NASH. In some instances where excessive alcohol intake is a contributor, FLD may also be termed alcoholic steatosis, or the more severe form alcoholic steatohepatitis (ASH). NASH is a progressive form, and generally severe form, of NAFLD where accumulation of excessive fat (steatosis) coexists with liver cell injury, inflammation and fibrosis, which eventually leads to cirrhosis and hepatocellular carcinoma.

Generally, the pathology of FLD is the intracytoplasmatic accumulation of triglycerides (neutral fats). In early stages of the disease, the hepatocytes present small fat vacuoles (liposomes) around the nucleus (microvesicular fatty change). Liver cells are filled with multiple fat droplets that do not displace the centrally located nucleus. In later stages, the size of the vacuoles increases, pushing the nucleus to the periphery of the cell, giving characteristic signet ring appearance (macrovesicular fatty change). These vesicles are well delineated and optically “empty” because fats dissolve during tissue processing. Large vacuoles may coalesce and produce fatty cysts, and other irreversible lesions. Macrovesicular steatosis is reportedly the most common form of FLD and is typically associated with alcohol, diabetes, obesity and corticosteroids. Acute fatty liver of pregnancy and Reye's syndrome are examples of severe liver disease caused by microvesicular fatty change. Generally, the diagnosis of steatosis is made when fat in the liver exceeds 5-10% by weight.

Defects in fatty acid metabolism may be responsible for the pathogenesis of FLD, which may be due to imbalance in energy consumption and its combustion, resulting in lipid storage. The transport of fatty acids from adipose tissue to the liver is increased. Without being bound by theory, it is believed herein that the compounds, compositions, and methods described herein are efficacious in treating FLD through CRP mediated pathways. The compounds, compositions, and methods described herein lower CRP levels, and those lower levels align with lower triglycerides, and an improved ratio of good (HDL) to bad (LDL) cholesterol.

Liver disease with extensive inflammation and a high degree of steatosis often progresses to more severe forms of the disease. Hepatocyte ballooning and necrosis of varying degrees are often present at this stage. Liver cell death and inflammatory responses lead to the activation of stellate cells, which play an important role in hepatic fibrosis. The further progression to cirrhosis may be influenced by the amount of fat and degree of steatohepatitis and by a variety of other sensitizing factors. In alcoholic FLD, the transition to cirrhosis related to continued alcohol consumption is well documented, but the process involved in nonalcoholic FLD is less clear. Left untreated, fatty liver disease, including NASH, reportedly progresses to hepatocellular carcinoma (HCC) in nearly all cases.

Chronic low grade systemic inflammation is also reportedly associated with aging and age related disabilities, and the term “inflammaging” has been coined to describe this inflammatory aging condition. Inflammaging reportedly contributes to many aging diseases and disabilities, such as, changes in body composition and physical ability, energy production, metabolic homeostasis, immune senescence, and cognitive ability.

In another embodiment, methods for treating and/or decreasing musculoskeletal decline are described herein.

Illustrative examples of musculoskeletal decline include, but are not limited to, muscle loss diseases, muscle atrophy, muscle repair, muscle recuperation, sarcopenia, speeding muscle building and accumulation, correcting muscle loss after trauma, injury, medical procedures, exercise, including heavy exercise, and the like.

Sarcopenia is a common aging phenotype characterized by a general loss of skeletal muscle mass and function leading to a loss of quality of life and ultimately death. Reportedly, 14% of adults between the age of 65 and 70 have one or more clinical signs of sarcopenia, and that increases to above 50% in persons greater than 80 years. A direct link between CRP and sarcopenia has been reported. Wallin-Larsson et al. “Mechanistic links underlying the impact of C-reactive protein on muscle mass in elderly.” Cellular Physiology and Biochemistry, 44(1):267-278 (2017). In vitro experiments showed that muscle cell size is negatively influenced by CRP via suppression of the muscle protein synthesis pathway, and moreover, elderly women who had elevated CRP levels also exhibited decreased muscle mass. Without being bound by theory, it is believed herein that a reduction in CRP may contribute to a slowing of sarcopenia during human aging.

In another embodiment, methods for treating and/or decreasing cognitive decline are described herein. Cognitive decline may lead to progressive impairments to memory, thinking, and behavior, which together may negatively impact the ability to function and carry out everyday activities. Cognitive decline often leads to a secondary wave of impairments including difficulties with language, decreased motivation, and most notably, emotional problems, including increased anger, irritability, and aggression. Cognitive decline is highly prevalent in neurodegenerative disorders such as Parkinson's disease, Huntington's disease, Alzheimer's disease, and the like. High circulating levels of CRP reportedly co-occur with neurodegenerative disorders. It is believed herein that the compounds, compositions, and methods described herein are efficacious in treating cognitive decline, including cognitive decline occurring in neurodegenerative diseases, by decreasing circulating levels of CRP.

It is also to be understood that the therapeutic efficacy of the compounds and compositions described herein, though initially arising from lowering CRP, may also derive efficacy through downstream consequences of CRP lowering. For example, CRP signaling through FcγRII may impact Smad3 signaling, which may initiate signaling of the mechanistic target of rapamycin (mTOR), also known as the mammalian target of rapamycin and FK506-binding protein 12-rapamycin-associated protein 1 (FRAP1). may be initiated by, which is indirectly controlled by. The In addition, but without being bound by theory, it is believed that the compounds and compositions described herein elicit their therapeutic effects by indirectly FcγRII may also cause decreasing NF-κB and/or ERK/p38 signaling. mTOR, NF-κB and/or ERK/p38 signaling each contribute to inflammation and fibrosis.

In another embodiment, AKG is provided as the free acid (α-ketoglutaric acid). In another embodiment, AKG is provided as a mono salt or bis salt. In other embodiments, AKG is provided as a monosodium salt, a disodium salt, a monopotassium salt, or a dipotassium salt. In yet further embodiments, AKG is provided as a monovalent or divalent salt with other cations described in the U.S. FDA Orange Book. Such cations include calcium, diolamine, lithium, lysine, magnesium, meglumine, olamine, tromethamine, and zinc. In further embodiments, salts of AKG are provided as anhydrous salts, hemihydrates, monohydrates, or dihydrates.

In another embodiment, the compositions and methods disclosed herein comprise AKG.

In another embodiment, the compositions and methods consists essentially of AKG. In another embodiment, the AKG in the compositions and methods consists essentially of a salt of AKG. In another embodiment, the AKG in the compositions and methods consists essentially of a calcium salt of AKG (Ca-AKG).

Further disclosed herein, in certain aspects, are compositions that comprise α-ketoglutarate salt. In another embodiment, α-ketoglutarate is provided as a calcium salt (Ca-AKG). In another embodiment, calcium α-ketoglutarate can be a hydrate calcium α-ketoglutarate. In another embodiment, calcium α-ketoglutarate can be a mono-hydrate calcium α-ketoglutarate. In another embodiment, calcium α-ketoglutarate can be hemi-hydrate calcium α-ketoglutarate. In another embodiment, calcium α-ketoglutarate can be anhydrous calcium α-ketoglutarate.

In another embodiment, the compositions disclosed herein comprise an ester of α-ketoglutarate. In another embodiment, the ester of α-ketoglutarate is a methyl ester of α-ketoglutarate. In another embodiment, the ester of α-ketoglutarate is a dimethyl ester of α-ketoglutarate. In another embodiment, the ester of α-ketoglutarate is an ethyl ester of α-ketoglutarate. In another embodiment, the ester of α-ketoglutarate is a diethyl ester of α-ketoglutarate.

Illustrative daily doses include administering about 1 mg to about 3,000 mg daily. Adult daily doses include administering about 100 mg to about 3,000 mg, or about 200 mg to about 3,000 mg, or about 300 mg to about 3,000 mg, or about 400 mg to about 3,000 mg, or about 500 mg to about 3,000 mg, or about 500 mg to about 2,500 mg, or about 500 mg to about 2,000 mg, or about 500 mg to about 1,500 mg, or about 500 mg to about 1,400 mg, or about 500 mg to about 1,300 mg, or about 500 mg to about 1,250 mg, or about 500 mg to about 1,200 mg, or about 500 mg to about 1,100 mg, or about 500 mg to about 1,000 mg daily. It is to be understood that pediatric and adolescent doses can be scaled based on subject body weight, where the adult daily doses are based on an average adult weight of about 70 kg.

In another embodiment, the composition is administered to the subject to achieve a therapeutically effective amount of Ca-AKG. In another embodiment, the composition comprises at least 100 mg of Ca-AKG. In another embodiment, the composition comprises at least 200 mg of Ca-AKG. In another embodiment, the composition comprises at least 250 mg of Ca-AKG. In another embodiment, the composition comprises at least 300 mg of Ca-AKG. In another embodiment, the composition comprises at least 350 mg of Ca-AKG. In another embodiment, the composition comprises at least 400 mg of Ca-AKG. In another embodiment, the composition comprises at least 450 mg of Ca-AKG. In another embodiment, the composition comprises at least 500 mg of Ca-AKG. In another embodiment, the composition comprises at least 550 mg of Ca-AKG. In another embodiment, the composition comprises at least 600 mg of Ca-AKG. In another embodiment, the composition comprises at least 650 mg of Ca-AKG. In another embodiment, the composition comprises at least 700 mg of Ca-AKG. In another embodiment, the composition comprises at least 750 mg of Ca-AKG. In another embodiment, the composition comprises at least 800 mg of Ca-AKG. In another embodiment, the composition comprises at least 850 mg of Ca-AKG. In another embodiment, the composition comprises at least 900 mg of Ca-AKG. In another embodiment, the composition comprises at least 950 mg of Ca-AKG. In another embodiment, the therapeutically effective amount of Ca-AKG is at least 1000 mg. It is to be understood that in each of the foregoing embodiments, the Ca-AKG may be the monohydrate.

Illustrative salts of AKG include, but are not limited to, alkali metal and alkali earth metal salts, such as calcium, and the like.

It is to be understood that the AKG or salt thereof can be in any and all crystalline forms, partially crystalline forms, and non-crystalline and/or amorphous forms. It is also to be understood that the AKG or salt thereof can be in any and all hydrated and/or solvated forms.

A composition comprising any of the active agents described herein may be formulated for sustained or slow release, also called timed release or controlled release. Such compositions may generally be administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of agent contained within a sustained release formulation depends upon the site of implantation, the rate and expected duration of release, and the nature of the condition, disease or disorder to be treated or prevented.

In another embodiment, AKG or Ca-AKG formulated as a sustained release tablet, as described in co-pending PCT International Application No. PCT/US20/36987, the disclosure of which is incorporated herein by reference in its entirety.

In another embodiment, the composition comprises a vitamin. In another embodiment, the composition further comprises vitamin A. In another embodiment, the amount of vitamin A is from 100 mcg to 3000 mcg. In another embodiment, the amount of vitamin A is from 200 mcg to 1000 mcg. In another embodiment, the amount of vitamin A is about 250 mcg. In another embodiment, the amount of vitamin A is about 450 mcg. In another embodiment, the amount of vitamin A is about 650 mcg. In another embodiment, the vitamin A is retinyl palmitate.

In another embodiment, the vitamin is vitamin D. In another embodiment, the amount of vitamin D is from 50 IU to 3000 IU. In another embodiment, the amount of vitamin D is from 200 IU to 2000 IU. In another embodiment, the amount of vitamin D is about 250 IU. In another embodiment, the amount of vitamin D is about 500 IU. In another embodiment, the amount of vitamin D is about 750 IU. In another embodiment, the vitamin D is cholecalciferol. In another embodiment, the composition further comprises vitamin D3. In another embodiment, the composition comprises about 12.5 mcg (500 IU) of vitamin D3.

In another embodiment, the composition is administered once daily. In another embodiment, the composition is administered twice daily. In another embodiment, the composition is administered three times daily. In another embodiment, the composition is administered once a week. In another embodiment, the composition is administered once a month. In another embodiment, the composition is administered to the subject for at least 3 months.

In another embodiment, the subject is a mammal. In another embodiment, the mammal is a human. In another embodiment, the mammal is a dog. In another embodiment, the mammal is a cat. In another embodiment, the mammal is livestock. In another embodiment, the subject is a male. In another embodiment, the subject is a female.

In another embodiment, the composition further comprises a pharmaceutically acceptable excipient selected from the group consisting of antiadherents, binders, coatings, colors, disintegrants, flavoring agents, antioxidants, sweetening agents, glidants, lubricants, preservatives, preservatives, sorbents, surfactants, vehicles. and combinations thereof.

In another embodiment, the composition further comprises a sweetener. In another embodiment, the sweetener is isomalt. In another embodiment, the composition further comprises wax. In another embodiment, the wax is carnauba wax and/or rice bran wax. In another embodiment, the composition further comprises one or more excipients. In another embodiment, the composition further comprises a first lubricant. In another embodiment, the first lubricant is stearic acid. In another embodiment, the composition comprises a second lubricant. In another embodiment, the second lubricant is magnesium stearate. In another embodiment, the composition comprises a glidant. In another embodiment, the glidant is silica. In another embodiment, the calcium alpha-ketoglutarate is calcium alpha-ketoglutarate monohydrate.

In another embodiment, the composition comprises 500-550 mg, or 525 mg of calcium alpha-ketoglutarate monohydrate, isomalt, vegetable wax (carnauba and/or rice bran), stearic acid, magnesium stearate, and silica.

In another embodiment, the composition comprises 500-550 mg of calcium alpha-ketoglutarate monohydrate; optionally 450 mcg of retinyl palmitate; and further comprising isomalt, vegetable wax (carnauba and/or rice bran), stearic acid, magnesium stearate, and silica.

In another embodiment, the composition comprises 500-550 mg of calcium alpha-ketoglutarate monohydrate; optionally 12.5 mcg (500 IU) of cholecalciferol; and further comprising isomalt, vegetable wax (carnauba and/or rice bran), stearic acid, magnesium stearate, and silica.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein with numerical limitations described herein, the term “about” generally refers to a reasonable range surrounding each number to which it is applied, such as ±5%, +3%, and/or ±2%.

As used herein with components of the compositions and methods described herein, the term “consists essentially of” is understood to indicate that the component is the major, primary, or sole contributor to the activity, physical property, and the like of the composition or and method. For example, compositions that consist essentially of Ca-AKG do not substantially include other compounds that provide a clinically significant decrease in circulating CRP levels. Similarly, methods that consist essentially of administering Ca-AKG do not substantially include the administration of other compounds that provide a clinically significant decrease in circulating CRP levels.

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In another embodiment, the solvates are amorphous, and in other embodiments, the solvates are crystalline.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and/or vehicles.

Illustrative formats for oral administration include tablets, capsules, elixirs, syrups, and the like.

As used herein, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “treat,” “treating” or “treatment,” as used herein, may include alleviating, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

some embodiments, the term “delay” or “delaying” as related to a disease or disorder may refer to a compound that, in a statistical sample, delays or postpones the occurrence of the disorder or condition in the treated sample relative to an untreated control sample, or delays the onset or reduces the severity of one or more symptoms of the disorder or condition relative to the untreated control sample.

As used herein, “host animal” or “subject” may refer to a mammal. In another embodiment, the mammal is a human. In another embodiment, the mammal is a cat. In another embodiment, the mammal is a dog. In another embodiment, the mammal is livestock. In another embodiment, the livestock is selected from the group consisting of cattle, sheep, goats, swine, poultry, and equine animals. In another embodiment, the host animal or subject is a male. In another embodiment, the host animal or subject is a female. In another embodiment, the human is at least 18 years, at least 20 years, at least 25 years, at least 30 years, at least 35 years, at least 40 years, at least 45 years, at least 50 years, at least 55 years, at least 60 years, at least 65 years, at least 70 years, at least 75 years, or at least 80 years of age.

Each publication cited herein is incorporated herein by reference in its entirety.

The following examples further illustrate specific embodiments of the invention; however, the following illustrative examples should not be interpreted in any way to limit the invention.

### EXAMPLES

METHOD EXAMPLE. Human Trial. Human volunteers are orally administered 1,000 mg of Ca-AKG once daily, in a divided does of 500 mg. Male volunteers are also orally administered 900 mcg of Vitamin A (retinyl palmitate) once daily. Female volunteers are also orally administered 25 mcg of Vitamin D once daily. Prior to the start of dosing, a baseline blood draw is obtained for hematology, chemistry, CRP measurement, and metabolite analyses. After the start of dosing, blood draws are obtained monthly.

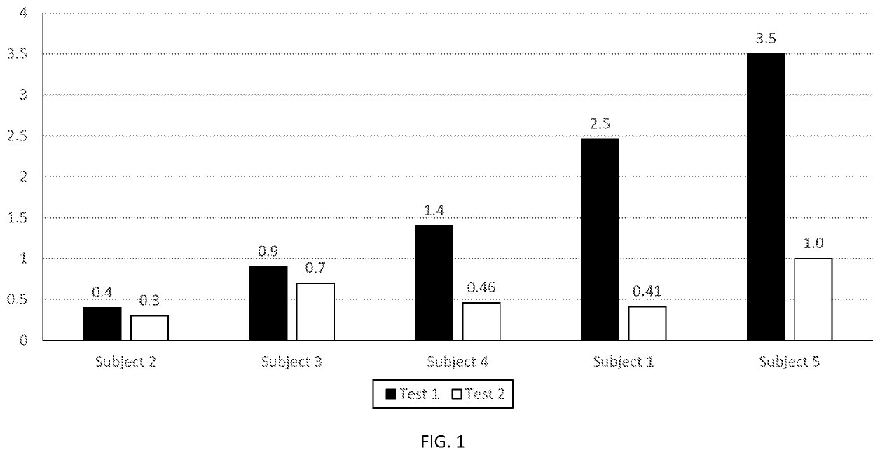

METHOD EXAMPLE. C-Reactive Protein (CRP) Human Data. Human test volunteers are administered two CaAKG tablets, each containing 500 mg of calcium alpha-ketoglutarate monohydrate, and optionally 900 mcg vitamin A (as retinyl palmitate) for males and 25 mcg vitamin D for females, for 3-7 months. The C-reactive protein (CRP) level (mg/L) is measured prior to treatment, and following treatment, by testing the corresponding blood samples from the test volunteers, as shown in FIG. 1.

Test 1 represents baseline CRP levels (t=0) in test subjects; Test 2 shows CRP levels following 3-7 months of treatment with compositions described herein comprising Ca-AKG. A decrease in CRP level was observed in all treated subjects. The average decrease was observed to be 54%, where even Subject 2, with a starting low level of CRP, showed a 25% decrease. The most dramatic decrease was observed in Subject 1, 84% after only 3 months of treatment. The data demonstrate that AKG, and salts thereof, are efficacious in treating CRP mediated diseases.

METHOD EXAMPLE. Blood chemistry. A human volunteer was orally administered 1,000 mg of Ca-AKG once daily, in a divided does of 500 mg, and 900 mcg of Vitamin A (retinyl palmitate) once daily. Prior to the start of dosing, a baseline blood draw is obtained for hematology, chemistry, CRP measurement, and metabolite analyses. After the start of dosing, blood draws are obtained monthly. The following results were obtained:

| Marker | Baseline | 10 Months | 12 Months |
|---|---|---|---|
| CRP (mg/L) | 4.9 | 2.5 | 0.4 |
| Cholesterol (mg/dL) | 184 | 184 | 181 |
| Triglycerides (mg/dL) | 85 | 49 | 52 |
| HDL (mg/dL) | 64 | 76 | 76 |
| LDL (mg/dL) | 103 | 98 | 95 |
| Total/HDL | 2.9 | 2.4 | 2.4 |
| Platelets (K/CUMM) | 359 | 392 | 332 |
| Glucose (mg/dL) | 95 | 104 | 99 |
| Alkaline phosphatase | 44 | 40 | 30 |
| (U/L) | | | |
| A1C (%) | 5.0 | 5.1 | 4.9 |
| PSA (ng/mL) | 7.5 | 10.1 | 7.7 |

The data demonstrate that AKG lowers CRP by more than 10-fold. In addition, AKG improves the levels of triglycerides and the relative levels of good versus bad cholesterol. In addition, AKG does not adversely affect other important biological markers, such as platelets, glucose, PSA, etc.

METHOD EXAMPLE. Cytokine Levels in Aged Mice. Female mice (18 months) are divided into treatment and untreated groups. Baseline levels of 30 inflammatory cytokines are measured. The treatment group is administered Ca-AKG, and aged to 29 months. Following treatment, levels of 30 inflammatory cytokines are measured from serum samples and compared to untreated controls. In untreated controls, the levels of most cytokines increase; however, Ca-AKG fed animals showed a general reduction compared to aged controls on average of 30 cytokines (Eotaxin, G-CSF, GM-CSF, IFN-γ, IL-1α, IL-1β, IL-2, IL-3, IL-4, IL-5, IL-6, IL-7, IL-9, IL-10, IL-12 (p40), IL-12 (p70), IL-13, IL-15, IL-17, IP-10, KC, LIX, M-CSF, MIG, MIP-1α, MIP-1β, MIP-2, RANTES, TNFα, VEGF). In addition, the levels of IL-3, IL-7, TNF-α, and MIP-1β were significantly lower (p<0.05) in the treatment group compared to aged controls, and moreover, were statistically indistinguishable the 18 month baseline. The data demonstrate that AKG, and salts thereof, are efficacious in treating inflammation.

1. A method for treating a CRP mediated disease in a host animal, the method comprising administering to the host animal a therapeutically effective amount of AKG, one or more salts thereof, or a combination of any of the foregoing, and optionally one or more diluents, excipients, or carriers, or a combination of any of the foregoing.

2. The method of claim 1 wherein the CRP mediated disease is musculoskeletal decline.

3. The method of claim 2 wherein the CRP mediated disease is a muscle atrophy, sarcopenia, or a combination thereof.

4. The method of claim 1 wherein the CRP mediated disease is a systemic inflammation.

5. The method of claim 4 wherein the CRP mediated disease is a cardiovascular disease associated with systemic inflammation.

6. The method of claim 5 wherein the cardiovascular disease is acute coronary syndrome, hemorrhagic stroke, or a combination thereof.

7. The method of claim 4 wherein the CRP mediated disease is age-related macular degeneration (AMD) associated with systemic inflammation.

8. The method of claim 4 wherein the CRP mediated disease is type 2 diabetes mellitus associated with systemic inflammation.

9. The method of claim 4 wherein the CRP mediated disease is fatty liver disease associated with systemic inflammation.

10. The method of claim 9 wherein the CRP mediated disease is fibrosis associated with fatty liver disease.

11. The method of claim 1 wherein the CRP mediated disease includes excessive cytokine signaling.

12. The method of claim 1 wherein the CRP mediated disease is cognitive decline.

13. The method of claim 12 wherein the CRP mediated disease is cognitive decline associated with a neurodegenerative disease.

14. The method of claim 1 wherein the therapeutically effective amount is an amount capable of decreasing circulating levels of CRP in the host animal.

15. The method of claim 1 wherein the therapeutically effective amount is a daily dose in the range from about 100 mg to about 3,500 mg of AKG, which is optionally divided.

16. The method of claim 1 wherein the therapeutically effective amount is a daily dose in the range from about 200 mg to about 3,500 mg of AKG, which is optionally divided.

17. (canceled)

18. The method of claim 1 wherein the composition comprises CaAKG.

19. The method of claim 1 further comprising administering one or more vitamins to the host animal.

20. The method of claim 19 wherein the vitamin is vitamin A.

21. The method of claim 19 wherein the vitamin is vitamin D.

22.-25. (canceled)
